SEQUENCE LISTING (5 sequences)

SEQ ID NO: 1 - DNA, 1014 bp, Homo sapiens
ggggcacgggccatgcctgaaccttcttccaccgccgcgccccgggcccgcgccccgccggccgcccgcactgggtcccgcgtcgcccccgccaccacaccgagccgcccagtacctcctcctcctgaaggcagagctgggggtccgtacggaggaacttcagaggctggaagcacgcttgaaaaagaaagggggcctgggctttgcaccagcttcaggattgcgtacttgtttcctcatccgtgaaatgggagcagctgaatgtgtctcctcgagttttcgagaaaaatgagggtctcactttgttgctcaggctgagctcgagctcctggcctcaagtgatcctcctgcttttggcctcccaaactgatgggattacaacgtggagccaccgtgttaaactcttagatttgactgctacaaaggtataaatgtggaactccatatatcagtgggttctgccccacttcttcctcccaggaattctccatcccagcctgctgctcctgtcttgcaccggggaacaaacatgtatcctggatgaaaataaaccaactcttgaaactgcaacccaagacattcagaagagataaggctacagtgaatgtggaaactttgcacgtgtttggatgtattgattgccttaactggcagctctttgcactgccagcttccctcaattacatgaggaaccacgagggggcaatgtcttcccgagcaataatagctgagtggtggtcttaggaccccgtggtggcccatgatgctagcactgtgtctttggccattcttgcctgtcatggagcatactccacagaagggcatggtcttcttggatcccggcatgggcccaacaaaaatgacaatgtagtcaatagttccactcagacctcacttaggagcaggtgaactatgttgtgtgatatcaggatgcccggagaacctctcctatccccacccagggaagcatggcttaattccaaataaagaatctgatttctttgtctttaagcc

SEQ ID NO: 2 - DNA, 779 bp, Homo sapiens
ggggcacgggccatgcctgaaccttcttccaccgccgcgccccgggcccgcgccccgccggccgcccgcactgggtcccgcgtcgcccccgccaccacaccgagccgcccagtacctcctcctcctgaaggcagagctgggggtccgtacggaggaacttcagaggctggaagcacgcttgaaaaagaaagggggcctgggctttgcaccagcttcaggattgcgtacttgtttcctcatccgtgaaatgggagcagctgaatgtgtctcctcgagttttcgaggggaacaaacatgtatcctggatgaaaataaaccaactcttgaaactgcaacccaagacattcagaagagataaggctacagtgaatgtggaaactttgcacgtgtttggatgtattgattgccttaactggcagctctttgcactgccagcttccctcaattacatgaggaaccacgagggggcaatgtcttcccgagcaataatagctgagtggtggtcttaggaccccgtggtggcccatgatgctagcactgtgtctttggccattcttgcctgtcatggagcatactccacagaagggcatggtcttcttggatcccggcatgggcccaacaaaaatgacaatgtagtcaatagttccactcagacctcacttaggagcaggtgaactatgttgtgtgatatcaggatgcccggagaacctctcctatccccacccagggaagcatggcttaattccaaataaagaatctgatttctttgtctttaagcc

SEQ ID NO: 3 - DNA, 860 bp, Homo sapiens
ggggcacgggccatgcctgaaccttcttccaccgccgcgccccgggcccgcgccccgccggccgcccgcactgggtcccgcgtcgcccccgccaccacaccgagccgcccagtacctcctcctcctgaagaaaaatgagggtctcactttgttgctcaggctgagctcgagctcctggcctcaagtgatcctcctgcttttggcctcccaaactgatgggattacaacgtggagccaccgtgttaaactcttagatttgactgctacaaaggtataaatgtggaactccatatatcagtgggttctgccccacttcttcctcccaggaattctccatcccagcctgctgctcctgtcttgcaccggggaacaaacatgtatcctggatgaaaataaaccaactcttgaaactgcaacccaagacattcagaagagataaggctacagtgaatgtggaaactttgcacgtgtttggatgtattgattgccttaactggcagctctttgcactgccagcttccctcaattacatgaggaaccacgagggggcaatgtcttcccgagcaataatagctgagtggtggtcttaggaccccgtggtggcccatgatgctagcactgtgtctttggccattcttgcctgtcatggagcatactccacagaagggcatggtcttcttggatcccggcatgggcccaacaaaaatgacaatgtagtcaatagttccactcagacctcacttaggagcaggtgaactatgttgtgtgatatcaggatgcccggagaacctctcctatccccacccagggaagcatggcttaattccaaataaagaatctgatttctttgtctttaagcc

SEQ ID NO: 4 - protein, 77 aa, Homo sapiens
MPEPSSTAAPRARAPPAARTGSRVAPATTPSRPVPPPPEGRAGGPYGGTSEAGSTLEKERGPGLCTSFRIAYLFPHP

SEQ ID NO: 5 - protein, 41 aa, Homo sapiens
MPEPSSTAAPRARAPPAARTGSRVAPATTPSRPVPPPPEEK

(19) United States
(12) Patent Application Publication
(10) Pub. No.: US 2008/0167245 A1
(43) Pub. Date: Jul. 10, 2008

(54) NOVEL METASTASIS SUPPRESSOR GENE ON HUMAN CHROMOSOME 8

(75) Inventors: Naoki Nihei, Tochigi (JP); J. Carl Barrett, Bethesda, MD (US); Natalay Kouprina, Potomac, MD (US); Vladimir Larionov, Potomac, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: The Government of the U.S.A., as represented by the Secretary , Dept. of Health & Human Services, Rockville, MD (US)

(21) Appl. No.: 12/045,585

(22) Filed: Mar. 10, 2008

Related U.S. Application Data
(63) Continuation of application No. 10/499,515, filed on Oct. 8, 2004, now abandoned.
(60) Provisional application No. 60/345,109, filed on Dec. 21, 2001.

(51) Int. Cl. A61K 31/70; C12N 15/11; C12N 15/00; C12N 5/06; C12Q 1/00; A61P 43/00; A61K 38/00; C07K 14/00; A61K 47/00

(52) U.S. Cl. 514/12; 536/23.5; 435/320.1; 435/325; 530/350; 514/773; 514/789; 514/44; 435/4

(57) ABSTRACT

An isolated or purified nucleic acid molecule consisting essentially of a nucleotide sequence encoding the metastasis suppressor gene located at p21-p12 on chromosome 8 of a human (Tey 1), a variant Tey 1, or a fragment of either of the foregoing comprising at least 455 contiguous nucleotides; an isolated or purified nucleic acid molecule consisting essentially of a nucleotide sequence that is complementary to a nucleotide sequence encoding Tey 1, a variant Tey 1, or a fragment of either of the foregoing comprising at least 455 contiguous nucleotides; a vector comprising any of the foregoing, wherein, when the isolated or purifies nucleic acid molecule consists essentially of a nucleotide sequence encoding Tey 1 or a variant thereof, the isolated or purified nucleic acid molecule is optionally part of an encoded fusion protein; a cell comprising and expressing any of the foregoing isolated or purified nucleic acid molecules, optionally in the form of a vector; an isolated or purified polypeptide molecule consisting essentially of an amino acid sequence encoding Tey 1, a variant Tey 1, or at least 6 contiguous amino acids of either of the foregoing; a conjugate or a fusion protein comprising a therapeutically or prophylactically active agent, and an excipient or an adjuvant, a method of treating cancer prophylactically or therapeutically in a mammal comprising administering to the mammal an effective amount of (a) an isolated or purified nucleic acid molecule encoding Tey 1, optionally in the for of a vector, or (b) an isolated or purified Tey 1 polypeptide, optionally in the form of a conjugate or fusion protein, whereupon the mammal is treated for the cancer prophylactically or therapeutically; a method of diagnosing cancer comprising (a) obtaining a test sample from the mammal, and (b) assaying the test sample for the level of Tey 1, wherein a decrease in the level of Tey 1 in the test sample as compared to the level of Tey 1, wherein a decrease in the level of Tey 1 in the test sample as compared to the level of Tey 1 in a control sample is diagnostic for the cancer, and a method of prognosticating cancer in a mammal comprising (a) obtaining a test sample from the mammal, and (b) assaying the test sample for the level of Tey 1, wherein an increase in the level of Tey 1 over time is indicative of a positive prognosis and a decrease in the level of Tey 1 over time is indicative of a negative prognosis.

### TECHNICAL FIELD OF THE INVENTION

The present invention relates to a metastasis suppressor gene located on chromosome 8 in humans and related vectors, host cells, polypeptides, compositions and methods of diagnosis, prognosis and treatment of cancer.

### BACKGROUND OF THE INVENTION

The American Cancer Society estimates the lifetime risk that an individual will develop cancer is 1 in 2 for men and 1 in 3 for women. The development of cancer, while still not completely understood, can be enhanced as a result of a variety of risk factors. For example, exposure to environmental factors (e.g., tobacco smoke) might trigger modifications in certain genes, thereby initiating cancer development. Alternatively, these genetic modifications may not require an exposure to environmental factors to become abnormal. Indeed, certain mutations (e.g., deletions, substitutions, etc.) can be inherited from generation to generation, thereby imparting an individual with a genetic predisposition to develop cancer.

Currently, the survival rates for many cancers are on the rise. One reason for this success is improvement in the detection of cancer at a stage at which treatment can be effective. Indeed, it has been noted that one of the most effective means to survive cancer is to detect its presence as early as possible. According to the American Cancer Society, the relative survival rate for many cancers would increase by about 15% if individuals participated in regular cancer screenings. Therefore, it is becoming increasingly useful to develop novel diagnostic tools to detect the cancer either before it develops or at an as early stage of development as possible.

One popular way of detecting cancer early is to analyze the genetic makeup of an individual to detect the presence or expression levels of a marker gene(s) related to the cancer. For example, there are various diagnostic methods that analyze a certain gene or a pattern of genes to detect cancers of the breast, tongue, mouth, colon, rectum, cervix, prostate, testis, and skin.

Prostate cancer is the most common non-cutaneous malignancy diagnosed in men in the United States, accounting for over 40,000 deaths annually (Parker et al., J. Clin. Cancer 46: 5 (1996)). While methods for early detection and treatment of prostate cancer have been forthcoming, there is an obvious need for improvement in this area. Therefore, the discovery of gene mutations which are good indicators of cancer, and more particularly prostate cancer, would be a tremendous step towards understanding the mechanisms underlying cancer and could offer a dramatic improvement in the ability of scientists to detect cancer and even to predict an individual's susceptibility to a particular type of cancer.

Much research has, in fact, been centered around establishing a genetic link to prostate cancer and studies have identified many recurring genetic changes associated with prostate cancer. These genetic changes include DNA hypermethylation, allelic loss, aneuploidy, aneusomy, various point mutations, and changes in protein expression level (e.g., E-cadherin/alpha-catenin). Researchers have also discovered losses and duplications in particular chromosomes or chromosome arms which are associated with prostate cancer (U.S. Pat. No. 5,925,519; Visakorpi, Ann. Chirur. Gynaec. 88:11-16 (1999)). In particular, losses of chromosomes 6q, 8p, 10q, 13q and 16q, and duplications of 7, 8q and Xq have be an associated with prostate cancer. Moreover, researchers have performed genetic epidemiological studies of affected populations and have identified various putative prostate cancer susceptibility loci, indicating that there is significant genetic heterogeneity in prostate cancer. These include Xq27-q28 (Xu et al., Nat. Genet. 20:175-179 (1998)) and 1q42-q43 (Gibbs et al., Am. J. Hum. Genet. 64:1087-1095 (1999); Berthon et al., Am. J. Hum. Genet. 62:1416-1424 (1998)).

One such potential prostate cancer susceptibility locus is the 1q24-q31 locus (flanked by D1S2883 and D1S422), which has been designated as HPC1 (due to its putative link to hereditary prostate cancer (HPC)). This HPC1 locus was identified in a genome-wide scan of families at high risk for prostate cancer (Smith et al., Science 274:1371-1374 (1996)). The HPC1 locus has been controversial, however, due to the fact that researchers have had difficulty duplicating the results of Smith et al. (De la Chapelle et al., Curr. Opin. Genet. Dev. 8:298-303 (1998)). In fact, some groups of researchers have found no linkage of the HPC1 locus to hereditary prostate cancer (Eeles et al., Am. J. Hum. Genet. 62:653-658 (1998); Thibodeau et al., Am. J. Hum. Genet. 61(suppl.):1733 (1997); McIndoe et al., Am. J. Hum. Genet. 61:347-353 (1997)), while others have found linkage in a very small fraction of high-risk prostate cancer families (Schleutker et al., Am. J. Hum. Genet. 61(suppl.):1711 (1997)). Further support for the linkage between the HPC1 locus and hereditary prostate cancer was revealed, however, via a combined Consortium analysis of 6 markers in the HPC1 region in 772 families segregating hereditary prostate cancer (see Xu et al., Am. J. Hum. Genet. 66:945-957 (2000)). In this regard, research findings concerning the HPC1 locus and its potential link to prostate cancer have been promising, but often nonconforming.

There also have been numerous reports of allelic loss of the p arm of chromosome 8 associated with prostate cancers (as high as 65% of prostate carcinomas) (see, e.g., Bookstein et al., U.S. Pat. No. 6,043,088). Numerous reports of genes associated with cancer and, more specifically, prostate cancer (see, e.g., An et al., U.S. Pat. Nos. 5,882,864; 5,972,615; 6,156,515; 6,171,796; and 6,218,529), on chromosome 8 (see, e.g., Ichikawa et al., Cancer Research 54: 2299-2302 (1994), and Kuramochi et al., The Prostate 31: 14-20 (1997)), in re human chromosome 8; Nihei et al., Genes, Chromosomes & Cancer 17: 260-268 (1996), and Ichikawa et al., Asian J. of Andrology 2(3): 167-171 (2000), in re metastasis suppressor gene at p21-p12 on chromosome 8; Ichikawa et al., The Prostate Supplement 6: 31-35 (1996), in re metastasis suppressor gene at 8p23-q12; Nihei et al., Proc. 90th Ann. Mtg. of the Amer. Assoc. Cancer Research 40: 105 (Abstract No. 699) (March 1999), in re metastasis suppressor gene at D8S131-D8S339 on the p arm of human chromosome 8; Sunwoo et al., Oncogene 18: 2651-2655 (1999), in re tumor suppressor at D8S264-D8S1788; Trapman et al., Cancer Research 54: 6061-6064 (1994), in re tumor suppressor gene at D8S87-D8S133; Konig et al., Urol. Res. 27(1): 3-8 (1999), in re tumor suppressor gene at 8p21 (see, also, Kagan et al., Oncogene 11: 2121-2126 (1995), and He et al., Genomics 43: 69-77 (1997)); Bookstein et al. (U.S. Pat. No. 6,043,088) in re prostate/colon tumor suppressor gene product (PTSG protein) from p22 region of chromosome 8 (see, also, Levy et al., Genes, Chromosomes & Cancer 24: 42-47 (1999), and Kagan et al., supra, in re homozygous deletions in this region); Cohen et al., International Patent Application No. WO 99/32644 in re PG1 gene from p23 region of chromosome 8; Wang et al., Genomics 60:1-11 (1999), in re tumor suppressor gene at 8p22-p23; and Oba et al., Cancer Genet. Cytogenet. 124: 20-26 (2001), in re two putative tumor suppressor genes at p21.1-p21.2 and p22-p21.3 (see, also, Suzuki et al., Genes, Chromosomes & Cancer 13; 168-174 (1995), on chromosome 8).

8p11 has been found to be a recurrent chromosomal breakpoint in prostate cancer cell lines (Pan et al., Genes, Chromosomes & Cancer 30: 187-195 (2001). It also has been reported that loss of 8p sequences may result from complex structural rearrangements involving chromosome 8, which sometimes includes i(8q) chromosome formation (Macoska et al., Cancer Research 55: 5390-5395 (1995), and Cancer Genet. Cytogenet. 120: 50-57 (2000)). Genetic changes at 8q in clinically organ-confined prostate cancer also have been noted (Fu et al., Urology 56: 880-885 (2000)). Differential expression of the gene GC84 at 8q11 has been associated with the progression of prostate cancer (Chang et al., Int. J. Cancer 83: 506-511 (1999)). 8p22 loss with 8c gain has been associated with poor outcome in prostate cancer (Macoska et al., Urology 55: 776-782 (2000)); see, also, Arbieva et al., Genome Research 10: 244-257 (2000)). Loss of 8p23 and 8q12-13 has been found to be associated with human prostate cancer (Perinchery et al., Int'l. J. Oncology 14: 495-500 (1999)). Gene amplification in 8q24 has been found to be associated with human prostate cancer (McGill et al., International Patent Application No. WO 96/20288). Mutations in the FEZ1 gene at 8p22 have been found to be associated with primary esophageal cancers and in a prostate cancer cell line (Ishii et al., PNAS USA 96: 3928-3933 (March 1999)).

The use of various gene sequences in the diagnosis and prognosis of cancer, specifically prostate cancer, also has been disclosed (see, e.g., An et al., supra; Russell et al., U.S. Pat. No. 5,861,248; Jenkins et al., European Patent Application EP 1 048 740; Bachner et al., U.S. Pat. No. 6,140,049; Ross et al., U.S. Pat. No. 5,994,071; and Jensen et al., U.S. Pat. No. 5,925,519).

Genes and gene products, which can be shown to have a strong association with cancer, such as prostate cancer, need to be identified. Such genes and gene products would lead directly to early, sensitive and accurate methods for detecting cancer or a predisposition to cancer in a mammal. Moreover, such methods would enable clinicians to monitor the onset and progression of cancer in an individual with greater sensitivity and accuracy, as well as the response of an individual to a particular treatment. The present invention provides such a gene and gene products, as well as related vectors, host cells, compositions and methods of use in the diagnosis, prognosis and treatment of cancer, in particular prostate cancer.

### BRIEF SUMMARY OF THE INVENTION

The present invention provides an isolated or purified nucleic acid molecule consisting essentially of a nucleotide sequence encoding the metastasis suppressor gene located at p21-p12 on chromosome 8 of a human, which has been named Tenni Yokusei 1 (Tey 1), or a fragment thereof comprising at least 455 contiguous nucleotides.

The present invention also provides an isolated or purified nucleic acid molecule consisting essentially of a nucleotide sequence encoding a variant Tey 1 or a fragment thereof comprising at least 455 contiguous nucleotides. The variant Tey 1 comprises one or more insertions, deletions, substitutions, and/or inversions and does not differ functionally from the corresponding unmodified Tey 1.

Still also provided by the present invention is an isolated or purified nucleic acid molecule consisting essentially of a nucleotide sequence that is complementary to a nucleotide sequence encoding Tey 1 or a fragment thereof comprising at least 455 contiguous nucleotides.

Thus, the present invention also provides an isolated or purified nucleic acid molecule consisting essentially of a nucleotide sequence that is complementary to a nucleotide sequence encoding a variant Tey 1.

In view of the above, the present invention further provides a vector comprising an above-described isolated or purified nucleic acid molecule. When the isolated or purified nucleic acid molecule consists essentially of a nucleotide sequence encoding Tey 1 or a variant thereof, the isolated or purified nucleic acid molecule is optionally part of an encoded fusion protein.

Also in view of the above, the present invention provides a cell comprising and expressing an above-described isolated or purified nucleic acid molecule, optionally in the form of a vector.

An isolated or purified polypeptide molecule consisting essentially of an amino acid sequence encoding Tey 1, a variant Tey 1, or at least 6 contiguous amino acids of either of the foregoing, which is optionally glycosylated, amidated, carboxylated, phosphorylated, esterified, N-acylated or converted into an acid addition salt, is also provided by the present invention. Thus, a conjugate or fusion protein comprising the isolated or purified polypeptide molecule or variant thereof and a therapeutically or prophylactically active agent is also provided as is a composition comprising the isolated or purified polypeptide molecule, optionally in the form of a conjugate or a fusion protein comprising a therapeutically or prophylactically active agent, and an excipient or an adjuvant.

In view of the above, a method of treating cancer prophylactically or therapeutically in a mammal is also provided. The method comprises administering to the mammal an effective amount of (a) an isolated or purified nucleic acid molecule encoding Tey 1, optionally in the form of a vector, or (b) an isolated or purified Tey 1 polypeptide, optionally in the form of a conjugate or fusion protein, whereupon the mammal is treated for the cancer prophylactically or therapeutically. Preferably, the cancer is prostate cancer.

Also in view of the above, a method of diagnosing cancer in a mammal is provided. The method comprises (a) obtaining a test sample from the mammal, and (b) assaying the test sample for the level of Tey 1, wherein a decrease in the level of Tey 1 in the test sample as compared to the level of Tey 1 in a control sample is diagnostic for the cancer.

A method of prognosticating cancer in a mammal is also provided. The method comprises (a) obtaining a test sample from the mammal, and (b) assaying the test sample for the level of Tey 1, wherein an increase in the level of Tey 1 over time is indicative of a positive prognosis and a decrease in the level of Tey 1 over time is indicative of a negative prognosis. The method of prognosticating can be used to assess the efficacy of treatment of the cancer.

### BRIEF DESCRIPTION OF THE FIGURES

FIG. 1 is the nucleotide sequence (SEQ ID NO: 1) of the cDNA of Tey 1, which is read 5′ to 3′ from top to bottom and left to right.

FIG. 2 is the nucleotide sequence (SEQ ID NO: 2) of an alternatively spliced cDNA of Tey 1, which is read 5′ to 3′ from top to bottom and left to right.

FIG. 3 is the deduced amino acid sequence (SEQ ID NO: 3) of the polypeptide encoded by the nucleotide sequence of SEQ ID NO: 1 or SEQ ID NO: 2, which is read N-terminus to C-terminus from top to bottom and left to right.

FIG. 4 is the nucleotide sequence (SEQ ID NO: 4) of an alternatively spliced cDNA of Tey 1, which is read 5′ to 3′ from top to bottom and left to right.

FIG. 5 is the deduced amino acid sequence (SEQ ID NO: 5) of the polypeptide encoded by the nucleotide sequence of SEQ ID NO: 4, which is read N-terminus to C-terminus from top to bottom and left to right.

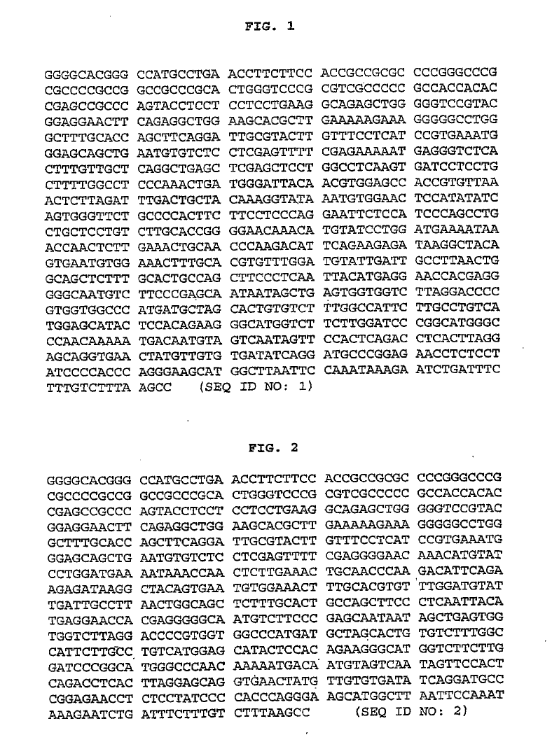

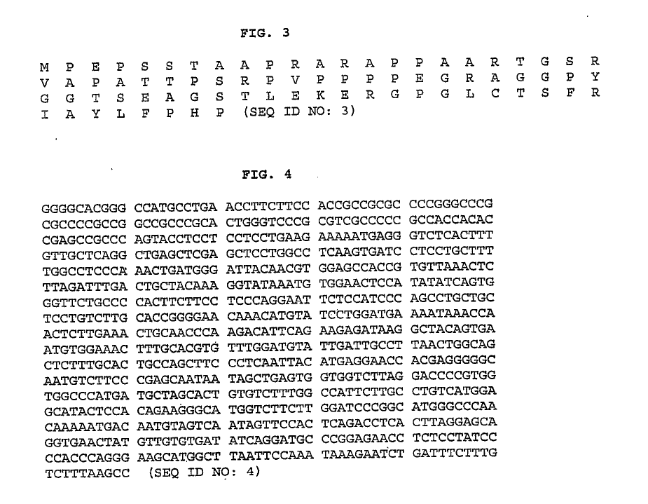

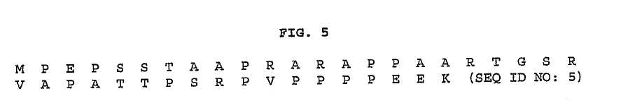

### DETAILED DESCRIPTION OF THE INVENTION

The present invention provides an isolated or purified nucleic acid molecule consisting essentially of a nucleotide sequence encoding Tey 1 or a fragment thereof comprising at least 455 (or at least 475, 500, 550 or 600 or more) contiguous nucleotides.

By “isolated” is meant the removal of a nucleic acid from its natural environment. By “purified” is meant that a given nucleic acid, whether one that has been removed from nature (including genomic DNA and mRNA) or synthesized (including cDNA) and/or amplified under laboratory conditions, has been increased in purity, wherein “purity” is a relative term, not “absolute purity.” “Nucleic acid molecule” is intended to encompass a polymer of DNA or RNA, i.e., a polynucleotide, which can be single-stranded or double-stranded and which can contain non-natural or altered nucleotides. Desirably, the isolated or purified nucleic acid molecule does not contain any introns or portions thereof.

Preferably, the isolated or purified nucleic acid molecule consists essentially of a nucleotide sequence encoding the amino acid sequence of SEQ ID NO: 3 or 5, (ii) consists essentially of the nucleotide sequence of SEQ ID NO: 1, 2 or 4 or a fragment thereof comprising at least 455 contiguous nucleotides, (iii) hybridizes under low stringency conditions to an isolated or purified nucleic acid molecule consisting essentially of the nucleotide sequence that is complementary to SEQ ID NO: 1, 2 or 4 or a fragment thereof comprising at least 455 (or at least 475, 500, 550 or 600 or more) contiguous nucleotides, or (iv) shares 50% (or 55%, 60%, 65%, 70%, 75% or 80% or more) or more identity with SEQ ID NO: 1, 2 or 4.

Also provided is an isolated or purified nucleic acid molecule consisting essentially of a nucleotide sequence encoding a variant Tey 1 or a fragment thereof comprising at least 455 (or at least 475, 500, 550 or 600 or more) contiguous nucleotides. The variant comprises one or more insertions, deletions, substitutions, and/or inversions. Desirably, the variant Tey 1 does not differ functionally from the corresponding unmodified Tey 1, such as that comprising SEQ ID NO: 3 or 5. Preferably, the variant Tey 1 is able to suppress metastasis of a highly metastatic prostatic tumor cell line in vivo at least about 75%, more preferably at least about 90% as well as the unmodified Tey 1 comprising SEQ ID NO: 3 or 5 as determined by in vivo assay. The manner in which the assay is carried out is not critical and can be conducted in accordance with methods known in the art. Preferably, the one or more substitution(s) results in the substitution of an amino acid of the encoded Tey 1 with another amino acid of equivalent mass, structure and/or charge.

The present invention also provides an isolated or purified nucleic acid molecule consisting essentially of a nucleotide sequence that is complementary to a nucleotide sequence encoding Tey 1 or a fragment thereof comprising at least 455 (or at least 475, 500, 550 or 600 or more) contiguous nucleotides. Such an isolated or purified nucleic acid molecule preferably (i) is complementary to a nucleotide sequence encoding the amino acid sequence of SEQ ID NO: 3 or 5, (ii) is complementary to the nucleotide sequence of SEQ ID NO: 1, 2 or 4 or a fragment thereof comprising at least 455 contiguous nucleotides, (iii) hybridizes under low stringency conditions to an isolated or purified nucleic acid molecule consisting essentially of SEQ ID NO: 1, 2 or 4 or a fragment thereof comprising at least 455 contiguous nucleotides, or (iv) shares 50% (or 55%, 60%, 65%, 70%, 75% or 80% or more) or more identity with the nucleotide sequence that is complementary to SEQ ID NO: 1.

Also provided is an isolated or purified nucleic acid molecule consisting essentially of a nucleotide sequence that is complementary to a nucleotide sequence encoding a variant Tey 1 as described above.

With respect to the above, one of ordinary skill in the art knows how to generate insertions, deletions, substitutions and/or inversions in a given nucleic acid molecule. See, for example, the references cited herein under “Example.” With respect to the above isolated or purified nucleic acid molecules, it is preferred that any such insertions, deletions, substitutions and/or inversions are introduced such that the metastasis suppressor activity is not compromised or is even enhanced. It is also preferred that the one or more substitution(s) result(s) in the substitution of an amino acid with another amino acid of equivalent size, shape and charge.

Also with respect to the above, “does not differ functionally from” is intended to mean that the variant Tey 1 has activity characteristic of the unmodified Tey 1. In other words, it can suppress metastasis of a tumor, particularly a prostatic tumor. However, the variant Tey 1 can be more or less active than the unmodified Tey 1 as desired in accordance with the present invention.

An indication that polynucleotide sequences are substantially identical is if two molecules selectively hybridize to each other under stringent conditions. The phrase “hybridizes to” refers to the selective binding of a single-stranded nucleic acid probe to a single-stranded target DNA or RNA sequence of complementary sequence when the target sequence is present in a preparation of heterogeneous DNA and/or RNA. “Stringent conditions” are sequence-dependent and will be different in different circumstances. Generally, stringent conditions are selected to be about 20° C. lower than the thermal melting point (Tm) for the specific sequence at a defined ionic strength and pH. The Tm is the temperature (under defined ionic strength and pH) at which 50% of the target sequence hybridizes to a perfectly matched probe.

For example, under stringent conditions, as that term is understood by one skilled in the art, hybridization is preferably carried out using a standard hybridization buffer at a temperature ranging from about 50° C. to about 75° C., even more preferably from about 60° C. to about 70° C., and optimally from about 65° C. to about 68° C. Alternately, formamide can be included in the hybridization reaction, and the temperature of hybridization can be reduced to preferably from about 35° C. to about 45° C., even more preferably from about 40° C. to about 45° C., and optimally to about 42° C. Desirably, formamide is included in the hybridization reaction at a concentration of from about 30% to about 50%, preferably from about 35% to about 45%, and optimally at about 40%. Moreover, optionally, the hybridized sequences are washed (if necessary to reduce non-specific binding) under relatively highly stringent conditions, as that term is understood by those skilled in the art. For instance, desirably, the hybridized sequences are washed one or more times using a solution comprising salt and detergent, preferably at a temperature of from about 50° C. to about 75° C., even more preferably at from about 60° C. to about 70° C., and optimally from about 65° C. to about 68° C. Preferably, a salt (e.g., such as sodium chloride) is included in the wash solution at a concentration of from about 0.01 M to about 1.0 M. Optimally, a detergent (e.g., such as sodium dodecyl sulfate) is also included at a concentration of from about 0.01% to about 1.0%. The following is an example of highly stringent conditions for a Southern hybridization in aqueous buffers (no formamide) (Sambrook and Russell, Molecular Cloning, 3rd Ed. SCHL Press (2001)):

Hybridization Conditions:

6×SSC or 6×SSPE

5×Denhardt's Reagent

1% SDS

100 ug/ml salmon sperm DNA

hybridization at 65-68° C.

Washing Conditions:

0.1×SSC/0.1% SDS

washing at 65-68° C.

Exemplary moderately stringent conditions, which allow for 25% mismatch, are as follows:

Hybridization Conditions:

5×SSC or 5×SSPE

5×Denhardt's Reagent

100 μg/ml salmon sperm DNA

hybridization at 50° C.

Washing Conditions:

1×SSC/0.1% SDS

washing at 55° C.

Exemplary low stringency conditions, which allow for 50% mismatch, are as follows:

Hybridization Conditions:

5×SSC or 5×SSPE

5×Denhardt's Reagent

100 μg/ml salmon sperm DNA

hybridization at 25° C.

Washing Conditions:

2×SSC/0.1% SDS

washing at 37° C.

In view of the above, “highly stringent conditions” allow for up to about 20% mismatch, preferably up to about 15% mismatch, more preferably up to about 10% mismatch, and most preferably less than about 5% mismatch, such as 4%, 3%, 2% or 1% mismatch. “At least moderately stringent conditions” preferably allow for up to about 45% mismatch, more preferably up to about 35% mismatch, and most preferably up to about 25% mismatch. “Low stringency conditions” preferably allow for up to 89% mismatch, more preferably up to about 70% mismatch, and most preferably up to about 50% mismatch. With respect to the preceding ranges of mismatch, 1% mismatch corresponds to one degree decrease in the melting temperature.

The above isolated or purified nucleic acid molecules also can be characterized in terms of “percentage of sequence identity.” In this regard, a given nucleic acid molecule as described above can be compared to a nucleic acid molecule encoding a corresponding gene (i.e., the reference sequence) by optimally aligning the nucleic acid sequences over a comparison window, wherein the portion of the polynucleotide sequence in the comparison window may comprise additions or deletions (i.e., gaps) as compared to the reference sequence, which does not comprise additions or deletions, for optimal alignment of the two sequences. The percentage of sequence identity is calculated by determining the number of positions at which the identical nucleic acid base occurs in both sequences, i.e., the number of matched positions, dividing the number of matched positions by the total number of positions in the window of comparison, and multiplying the result by 100 to yield the percentage of sequence identity. Optimal alignment of sequences for comparison may be conducted by computerized implementations of known algorithms (e.g., GAP, BESTFIT, FASTA, and TFASTA in the Wisconsin Genetics Software Package, Genetics Computer Group (GCG), 575 Science Dr., Madison, Wis., or BlastN and BlastX available from the National Center for Biotechnology Information, Bethesda, Md.), or by inspection. Sequences are typically compared using BESTFIT or BlastN with default parameters.

“Significant sequence identity” means that preferably at least 45%, more preferably at least 50%, and most preferably at least 55% (such as 60%, 65%, 70%, 75%, 80%, 85%, 90%, 95% or more) of the sequence of a given nucleic acid molecule is identical to a given reference sequence. Typically, two polypeptides are considered to have “substantial sequence identity” if at least 45%, preferably at least 60%, more preferably at least 90%, and most preferably at least 95% (such as 96%, 97%, 98% or 99%) of the amino acids of which the polypeptides are comprised are identical to or represent conservative substitutions of the amino acids of a given reference sequence.

One of ordinary skill in the art will appreciate, however, that two polynucleotide sequences can be substantially different at the nucleic acid level, yet encode substantially similar, if not identical, amino acid sequences, due to the degeneracy of the genetic code. The present invention is intended to encompass such polynucleotide sequences.

While the above-described nucleic acid molecules can be isolated or purified, alternatively they can be synthesized. Methods of nucleic acid synthesis are known in the art. See, e.g., the references cited herein under “Example.”

The above-described nucleic acid molecules can be used, in whole or in part (i.e., as fragments or primers), to identify and isolate related genes from humans (and other mammals) for use in the context of the present inventive methods using conventional means known in the art. See, for example, the references cited herein under “Example.” It will be possible to identify highly related Tey 1 nucleic acids using portions of the sequence given in SEQ ID NO:1, for example.

In view of the above, the present invention also provides a vector comprising an above-described isolated or purified nucleic acid molecule, optionally as part of an encoded fusion protein. A nucleic acid molecule as described above can be cloned into any suitable vector and can be used to transform or transfect any suitable host. The selection of vectors and methods to construct them are commonly known to persons of ordinary skill in the art and are described in general technical references (see, in general, “Recombinant DNA Part D,” Methods in Enzymology, Vol. 153, Wu and Grossman, eds., Academic Press (1987) and the references cited herein under “Example”). Desirably, the vector comprises regulatory sequences, such as transcription and translation initiation and termination codons, which are specific to the type of host (e.g., bacterium, fungus, plant or animal) into which the vector is to be introduced, as appropriate and taking into consideration whether the vector is DNA or RNA. Preferably, the vector comprises regulatory sequences that are specific to the genus of the host. Most preferably, the vector comprises regulatory sequences that are specific to the species of the host.

Constructs of vectors, which are circular or linear, can be prepared to contain an entire nucleic acid sequence as described above or a portion thereof ligated to a replication system functional in a prokaryotic or eukaryotic host cell. Replication systems can be derived from Co1E1, 2 mμ plasmid, λ, SV40, bovine papilloma virus, and the like.

In addition to the replication system and the inserted nucleic acid, the construct can include one or more marker genes, which allow for selection of transformed or transfected hosts. Marker genes include biocide resistance, e.g., resistance to antibiotics, heavy metals, etc., complementation in an auxotrophic host to provide prototrophy, and the like.

Suitable vectors include those designed for propagation and expansion or for expression or both. A preferred cloning vector is selected from the group consisting of the pUC series, the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λ EMBL4, and λ NM1149, also can be used. Examples of plant expression vectors include pBI101, pBI101.2, pBI101.3, pBI121 and pBIN19 (Clontech). Examples of animal expression vectors include pEUK-C1, pMAM and pMAMneo (Clontech).

An expression vector can comprise a native or normative promoter operably linked to an isolated or purified nucleic acid molecule as described above. The selection of promoters, e.g., strong, weak, inducible, tissue-specific and developmental-specific, is within the skill in the art. Similarly, the combining of a nucleic acid molecule as described above with a promoter is also within the skill in the art.

Optionally, the isolated or purified nucleic acid molecule can be part of an encoded fusion protein. The generation of fusion proteins is within the ordinary skill in the art (see, e.g., references cited under “Example”) and can involve the use of restriction enzyme or recombinational cloning techniques (see, e.g., Gateway™ (Invitrogen, Carlsbad, Calif.). See, also, U.S. Pat. No. 5,314,995.

Also in view of the above, the present invention provides a host cell comprising and expressing an isolated or purified nucleic acid molecule, optionally in the form of a vector, as described above. Examples of host cells include, but are not limited to, a human cell, a human cell line, E. coli (e.g., E. coli TB-1, TG-2, DH5α, XL-Blue MRF′ (Stratagene), SA2821 and Y1090), B. subtilis, P. aerugenosa, S. cerevisiae, N. crassa, insect cells (e.g., Sf9, Ea4) and others set forth herein below.

The present invention further provides an isolated or purified polypeptide molecule consisting essentially of an amino acid sequence encoding Tey 1 or at least 6 (or at least 10, 20, 30, 40, 50, 60, 70, 80, 90, or 100 or more) contiguous amino acids of Tey 1, which is optionally glycosylated, amidated, carboxylated, phosphorylated, esterified, N-acylated or converted into an acid addition salt. Methods of protein modification (e.g., glycosylation, amidation, carboxylation, phosphorylation, esterification, N-acylation, and conversion into acid addition salts) are known in the art.

Also provided is an isolated or purified polypeptide molecule consisting essentially of an amino acid sequence encoding a variant Tey 1 or at least 6 (or at least 10, 20, 30, 40, 50, 60, 70, 80, 90, or 100 or more) contiguous amino acids of a variant Tey 1, which is optionally glycosylated, amidated, carboxylated, phosphorylated, esterified, N-acylated, converted into an acid addition salt.

The polypeptide preferably comprises an amino end and a carboxyl end. The polypeptide can comprise D-amino acids, L-amino acids or a mixture of D- and L-amino acids. The D-form of the amino acids, however, is particularly preferred since a polypeptide comprised of D-amino acids is expected to have a greater retention of its biological activity in vivo, given that the D-amino acids are not recognized by naturally occurring proteases.

The polypeptide can be prepared by any of a number of conventional techniques. The polypeptide can be isolated or purified from a naturally occurring source or from a recombinant source. Recombinant production is preferred. For instance, in the case of recombinant polypeptides, a DNA fragment encoding a desired peptide can be subcloned into an appropriate vector using well-known molecular genetic techniques (see, e.g., Maniatis et al., Molecular Cloning: A Laboratory Manual, 2nd ed. (Cold Spring Harbor Laboratory, 1982); Sambrook et al., Molecular Cloning: A Laboratory Manuals 2^{nd} ed. (Cold Spring Harbor Laboratory, 1989). The fragment can be transcribed and the polypeptide subsequently translated in vitro. Commercially available kits also can be employed (e.g., such as manufactured by Clontech, Palo Alto, Calif.; Amersham Pharmacia Biotech Inc., Piscataway, N.J.; InVitrogen, Carlsbad, Calif., and the like). The polymerase chain reaction optionally can be employed in the manipulation of nucleic acids.

Alterations of the native amino acid sequence to produce variant polypeptides can be done by a variety of means known to those skilled in the art. For instance, site-specific mutations can be introduced by ligating into an expression vector a synthesized oligonucleotide comprising the modified site. Alternately, oligonucleotide-directed site-specific mutagenesis procedures can be used such as disclosed in Walder et al., Gene 42: 133 (1986); Bauer et al., Gene 37: 73 (1985); Craik, Biotechniques, 12-19 (January 1995); and U.S. Pat. Nos. 4,518,584 and 4,737,462.

With respect to the above isolated or purified polypeptides, it is preferred that any such insertions, deletions and/or substitutions are introduced such that the metastasis suppressor activity is not compromised or is even enhanced. It is also preferred that the one or more substitution(s) result(s) in the substitution of an amino acid with another amino acid of equivalent mass, structure and charge.

Any appropriate expression vector (e.g., as described in Pouwels et al., Cloning Vectors: A Laboratory Manual (Elsevier, N.Y.: 1985)) and corresponding suitable host can be employed for production of recombinant polypeptides. Expression hosts include, but are not limited to, bacterial species within the genera Escherichia, Bacillus, Pseudomonas, Salmonella, mammalian or insect host cell systems including baculovirus systems (e.g., as described by Luckow et al., Bio/Technology 6: 47 (1988)), and established cell lines such as the COS-7, C127, 3T3, CHO, HeLa, BHK cell line, and the like. The ordinarily skilled artisan is, of course, aware that the choice of expression host has ramifications for the type of polypeptide produced. For instance the glycosylation of polypeptides produced in yeast or mammalian cells (e.g., COS-7 cells) will differ from that of polypeptides produced in bacterial cells, such as Escherichia coli.

Alternately, the polypeptide (including the variant polypeptides) can be synthesized using standard peptide synthesizing techniques well-known to those of ordinary skill in the art (e.g., as summarized in Bodanszky, Principles of Peptide Synthesis, (Springer-Verlag, Heidelberg: 1984)). In particular, the polypeptide can be synthesized using the procedure of solid-phase synthesis (see, e.g., Merrifield, J. Am. Chem. Soc. 85: 2149-54 (1963); Barany et al., Int. J. Peptide Protein Res. 30: 705-739 (1987); and U.S. Pat. No. 5,424,398). If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The polypeptide-containing mixture can then be extracted, for instance, with dimethyl ether, to remove non-peptidic organic compounds, and the synthesized polypeptide can be extracted from the resin powder (e.g., with about 25% w/v acetic acid). Following the synthesis of the polypeptide, further purification (e.g., using high performance liquid chromatography (HPLC)) optionally can be done in order to eliminate any incomplete polypeptides or free amino acids. Amino acid and/or HPLC analysis can be performed on the synthesized polypeptide to validate its identity. For other applications according to the invention, it may be preferable to produce the polypeptide as part of a larger fusion protein, such as by the methods described herein or other genetic means, or as part of a larger conjugate, such as through physical or chemical conjugation, as known to those of ordinary skill in the art and described herein.

If desired, the polypeptides of the invention (including variant polypeptides) can be modified, for instance, by glycosylation, amidation, carboxylation, or phosphorylation, or by the creation of acid addition salts, amides, esters, in particular C-terminal esters, and N-acyl derivatives of the polypeptides of the invention. The polypeptides also can be modified to create polypeptide derivatives by forming covalent or noncovalent complexes with other moieties in accordance with methods known in the art. Covalently-bound complexes can be prepared by linking the chemical moieties to functional groups on the side chains of amino acids comprising the polypeptides, or at the N- or C-terminus.

Thus, in this regard, the present invention also provides a fusion protein and a conjugate comprising an above-described isolated or purified polypeptide molecule or fragment thereof and a therapeutically or prophylactically active agent. “Prophylactically” as used herein does not necessarily mean prevention, although prevention is encompassed by the term. Prophylactic activity also can include lesser effects, such as inhibition of the spread of cancer. Preferably, the active agent is an anti-cancer agent. Methods of conjugation are known in the art. In addition, conjugate kits are commercially available. For examples of methods of conjugation and conjugates see, e.g., Hermanson, G. T., Bioconjugate Techniques, 1996, Academic Press, San Diego, Calif.; U.S. Pat. Nos. 6,013,779; 6,274,552 and 6,080,725 and Ragupathi et al., Glycoconjugate Journal 15: 217-221 (1998).

The present invention also provides a composition comprising an above-described isolated or purified polypeptide molecule, optionally in the form of a conjugate or a fusion protein comprising a prophylactically or therapeutically active agent, and an excipient or an adjuvant. Excipients and adjuvants are well-known in the art, and are readily available. The choice of excipient/adjuvant will be determined in part by the particular route of administration and whether a nucleic acid molecule or a polypeptide molecule (or conjugate or fusion protein thereof) is being administered. Accordingly, there is a wide variety of suitable formulations for use in the context of the present invention, and the invention expressly provides a pharmaceutical composition that comprises an active agent of the invention and a pharmaceutically acceptable excipient/adjuvant. The following methods and excipients/adjuvants are merely exemplary and are in no way limiting.

Formulations suitable for oral administration can consist of (a) liquid solutions, such as an effective amount of the compound dissolved in diluent, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth. Pastilles can comprise the active ingredient in an inert base, such as gelatin and glycerin, or sucrose and acacia, emulsions, gels, and the like containing, in addition to the active ingredient, such excipients/carriers as are known in the art.

An active agent of the present invention, either alone or in combination with other suitable components, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Additionally, active agents of the present invention can be made into suppositories by mixing with a variety of bases such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate. Further suitable formulations are found in Remington's Pharmaceutical Sciences, 17th ed., (Mack Publishing Company, Philadelphia, Pa.: 1985), and methods of drug delivery are reviewed in, for example, Langer, Science 249: 1527-1533 (1990).

In view of the above, the present invention provides a method of treating cancer prophylactically or therapeutically in a mammal. The method comprises administering to the mammal an effective amount of (a) an isolated or purified nucleic acid molecule encoding Tey 1, optionally in the form of a vector, or (b) an isolated or purified Tey 1 polypeptide, optionally in the form of a conjugate or fusion protein, whereupon the mammal is treated for the cancer prophylactically or therapeutically. Preferably, the cancer is prostate cancer.

The anti-cancer agent can be a chemotherapeutic agent, e.g., a polyamine or an analogue thereof. Examples of therapeutic polyamines include those set forth in U.S. Pat. Nos. 5,880,161, 5,541,230 and 5,962,533, Saab et al., J. Med. Chem. 36: 2998-3004 (1993), Bergeron et al., J. Med. Chem. 37(21): 3464-3476 (1994), Casero et al., Cancer Chemother. Pharmacol 36: 69-74 (1995), Bernacki et al., Clin. Cancer Res. 1: 847-857 (1995); Bergeron et al., J. Med. Chem. 40: 1475-1494 (1997); Gabrielson et al., Clinical Cancer Res. 5: 1638-1641 (1999), and Bergeron et al., J. Med. Chem. 43: 224-235 (2000), which can be administered alone or in combination with other active agents, such as anti-cancer agents, e.g., cis-diaminedichloroplatinum (II) and 1,3-bis(2-chloroethyl)-1-nitrosourea.

Preferred routes of administration in the first embodiment of the method of treating cancer include intratumoral and peritumoral routes of administration. A preferred manner of administering a separate anti-cancer agent is by targeting to a cancer cell. In this regard, examples of cancer-specific, cell-surface molecules include placental alkaline phosphatase (testicular and ovarian cancer), pan carcinoma (small cell lung cancer), polymorphic epithelial mucin (ovarian cancer), prostate-specific membrane antigen, α-fetoprotein, B-lymphocyte surface antigen (B-cell lymphoma), truncated EGFR (gliomas), idiotypes (B-cell lymphoma), gp95/gp97 (melanoma), N-CAM (small cell lung carcinoma), cluster w4 (small cell lung carcinoma), cluster 5A (small cell carcinoma), cluster 6 (small cell lung carcinoma), PLAP (seminomas, ovarian cancer, and non-small cell lung cancer), CA-125 (lung and ovarian cancers), ESA (carcinoma), CD19, 22 or 37 (B-cell lymphoma), 250 kD proteoglycan (melanoma), P55 (breast cancer), TCR-IgH fusion (childhood T-cell leukemia), blood group A antigen in B or O type individual (gastric and colon tumors), and the like. See, e.g., U.S. Pat. No. 6,080,725 for other examples.

Examples of cancer-specific, cell-surface receptors include erbB-2, erbB-3, erbB-4, IL-2 (lymphoma and leukemia), IL-4 (lymphoma and leukemia), IL-6 (lymphoma and leukemia), MSH (melanoma), transferrin (gliomas), tumor vasculature integrins, and the like. Preferred cancer-specific, cell-surface receptors include erbB-2 and tumor vasculature integrins, such as CD11a, CD11b, CD11c, CD18, CD29, CD51, CD61, CD66d, CD66e, CD106, and CDw145.

There are a number of antibodies to cancer-specific, cell-surface molecules and receptors that are known. C46 Ab (Amersham) and 85A12 Ab (Unipath) to carcino-embryonic antigen, H17E2 Ab (ICRF) to placental alkaline phosphatase, NR-LU-10 Ab (NeoRx Corp.) to pan carcinoma, HMFC1 Ab (ICRF) to polymorphic epithelial mucin, W14 Ab to B-human chorionic gonadotropin, RFB4 Ab (Royal Free Hospital) to B-lymphocyte surface antigen, A33 Ab (Genex) to human colon carcinoma, TA-99 Ab (Genex) to human melanoma, antibodies to c-erbB2 (JP 7309780, JP 8176200 and JP 7059588), and the like. ScAbs can be developed, based on such antibodies, using techniques known in the art (see for example, Bind et al., Science 242: 423-426 (1988), and Whitlow et al., Methods 2(2): 97-105 (1991)).

Generally, when Tey 1 (or a conjugate or fusion protein thereof) is administered to an animal, such as a mammal, in particular a human, it is desirable that Tey 1 be administered in a dose of from about 1 to about 1,000 μg/kg body weight/treatment when given parenterally. Higher or lower doses may be chosen in appropriate circumstances. For instance, the actual dose and schedule can vary depending on whether the composition is administered in combination with other pharmaceutical compositions, or depending on interindividual differences in pharmacokinetics, drug disposition, and metabolism. One skilled in the art easily can make any necessary adjustments in accordance with the necessities of the particular situation.

Those of ordinary skill in the art can easily make a determination of the amount of an above-described isolated and purified nucleic acid molecule to be administered to an animal, such as a mammal, in particular a human. The dosage will depend upon the particular method of administration, including any vector or promoter utilized. For purposes of considering the dose in terms of particle units (pu), also referred to as viral particles, it can be assumed that there are 100 particles/pfu (e.g., 1×10¹² pfu is equivalent to 1×10¹⁴ pu). An amount of recombinant virus, recombinant DNA vector or RNA genome sufficient to achieve a tissue concentration of about 10² to about 10¹² particles per ml is preferred, especially of about 10⁶ to about 10¹⁰ particles per ml. In certain applications, multiple daily doses are preferred. Moreover, the number of doses will vary depending on the means of delivery and the particular recombinant virus, recombinant DNA vector or RNA genome administered.

A targeting moiety also can be used in the contact of a cell with an above-described isolated or purified nucleic acid molecule. In this regard, any molecule that can be linked with the therapeutic nucleic acid directly or indirectly, such as through a suitable delivery vehicle, such that the targeting moiety binds to a cell-surface receptor, can be used. The targeting moiety can bind to a cell through a receptor, a substrate, an antigenic determinant or another binding site on the surface of the cell. Examples of a targeting moiety include an antibody (i.e., a polyclonal or a monoclonal antibody), an immunologically reactive fragment of an antibody, an engineered immunoprotein and the like, a protein (target is receptor, as substrate, or regulatory site on DNA or RNA), a polypeptide (target is receptor), a peptide (target is receptor), a nucleic acid, which is DNA or RNA (i.e., single-stranded or double-stranded, synthetic or isolated and purified from nature; target is complementary nucleic acid), a steroid (target is steroid receptor), and the like. Analogs of targeting moieties that retain the ability to bind to a defined target also can be used. In addition, synthetic targeting moieties can be designed, such as to fit a particular epitope. Alternatively, the therapeutic nucleic acid can be encapsulated in a liposome comprising on its surface the targeting moiety.

The targeting moiety includes any linking group that can be used to join a targeting moiety to, in the context of the present invention, an above-described nucleic acid molecule. It will be evident to one skilled in the art that a variety of linking groups, including bifunctional reagents, can be used. The targeting moiety can be linked to the therapeutic nucleic acid by covalent or non-covalent bonding. If bonding is non-covalent, the conjugation can be through hydrogen bonding, ionic bonding, hydrophobic or van der Waals interactions, or any other appropriate type of binding.

Also in view of the above, the present invention provides a method of diagnosing cancer in a mammal. The method comprises (a) obtaining a test sample from the mammal, and (b) assaying the test sample for the level of Tey 1. A decrease in the level of Tey 1 in the test sample as compared to the level of Tey 1 in a control sample is diagnostic for the cancer.

The test sample used in conjunction with the invention can be any of those typically used in the art. For example, the test sample can be tissue. Typically, the tissue is metastatic (e.g., cancerous) and is obtained by means of a biopsy. Such tissue can include bone marrow, lymph nodes, skin, and any organ that may develop cancerous cells. Preferably, however, the test sample is taken from a source in which secreted proteins will be most prevalent. Accordingly, the test sample is preferably serum, wherein the serum is obtained from methods known in the art, such as a blood sample.

A number of assays are contemplated for use in the present inventive methods of diagnosing cancer. A number of these assays are described in Sambrook et al., Molecular Cloning: A Laboratory Manual, 2nd Ed., Cold Spring Harbor Press, Cold Spring Harbor, N.Y., 1989. Microarrays, such as those described in U.S. Pat. Nos. 6,197,506 and 6,040,138, also can be used to detect and quantify Tey 1. It will be understood that the type of assay used will depend on whether DNA, RNA or a protein (or a polypeptide thereof) is being assayed.

As used herein, the term “increased level” can be defined as detecting Tey 1 in a mammal at a level above that which is considered normal. For example, the level of Tey 1 in a test sample is increased when the copy number of the gene encoding the Tey 1 is greater than 1, the mRNA encoding Tey 1 is about 0.001-1%, or Tey 1 (or a polypeptide thereof) is detected in an amount of about 1-10,000 ng/ml.

When a nucleic acid (i.e., DNA or RNA) is assayed, various assays can be used to measure the presence and/or level of nucleic acid present. For example, when only the detection of Tey 1 is necessary to diagnose effectively the cancer, assays including PCR and microarray analysis can be used. In certain embodiments, it will be necessary to detect the quantity of Tey 1 present. In these embodiments, it will be advantageous to use various hybridization techniques known in the art that can effectively measure the level of Tey 1 in a test sample. When the Tey 1 comprises DNA, such hybridization techniques can include, for example, Southern hybridization (i.e., a Southern blot), in situ hybridization and microarray analysis. Similarly, when the Tey 1 comprises RNA, Northern hybridization (i.e., a Northern blot), in situ hybridization and microarray analysis are contemplated.

It will be understood that, in such assays, a nucleic acid sequence that specifically binds to or associates with a nucleic acid encoding Tey 1, whether DNA or RNA, can be attached to a label for determining hybridization. A wide variety of appropriate labels are known in the art, including fluorescent, radioactive, and enzymatic labels as well as ligands, such as avidin/biotin, which are capable of being detected. Preferably, a fluorescent label or an enzyme tag, such as urease, alkaline phosphatase or peroxidase, is used instead of a radioactive or other environmentally undesirable label. In the case of enzyme tags, calorimetric indicator substrates are known which can be employed to provide a detection means visible to the human eye or spectrophotometrically to identify specific hybridization with complementary Tey 1 nucleic acid-containing samples.

When a nucleic acid encoding the Tey 1 is amplified in the context of a diagnostic application, the nucleic acid used as a template for amplification is isolated from cells contained in the test sample, according to standard methodologies. (Sambrook et al., Molecular Cloning: A Laboratory Manual, 2nd Ed., Cold Spring Harbor Press, Cold Spring Harbor, N.Y., 1989). The nucleic acid can be genomic DNA or fractionated or whole cell RNA. Where RNA is used, it can be desirable to convert the RNA to cDNA.

In a typical amplification procedure, pairs of primers that selectively hybridize to nucleic acids corresponding to Tey 1 are contacted with the nucleic acid under conditions that permit selective hybridization. Once hybridized, the nucleic acid-primer complex is contacted with one or more enzymes that facilitate template-dependent nucleic acid synthesis. Multiple rounds of amplification, also referred to as “cycles,” are conducted until a sufficient amount of amplification product is produced.

Various template-dependent processes are available to amplify the Tey 1 present in a given test sample. As with the various assays, a number of these processes are described in Sambrook et al. (1989), supra. One of the best-known amplification methods is the polymerase chain reaction (PCR). Similarly, a reverse transcriptase PCR (RT-PCR) can be used when it is desired to convert mRNA into cDNA. Alternative methods for reverse transcription utilize thermostable DNA polymerases and are described in WO 90/07641, for example.

Other methods for amplification include the ligase chain reaction (LCR), which is disclosed in U.S. Pat. No. 4,883,750; isothermal amplification, in which restriction endonucleases and ligases are used to achieve the amplification of target molecules that contain nucleotide 5′-[alpha-thio]-triphosphates in one strand (Walker et al., Proc. Natl. Acad. Sci. USA 89: 392-396 (1992)); strand displacement amplification (SDA), which involves multiple rounds of strand displacement and synthesis, i.e., nick translation; and repair chain reaction (RCR), which involves annealing several probes throughout a region targeted for amplification, followed by a repair reaction in which only two of the four bases are present. The other two bases can be added as biotinylated derivatives for easy detection. Target-specific sequences also can be detected using a cyclic probe reaction (CPR). In CPR, a probe having 3′ and 5′ sequences of non-specific DNA and a middle sequence of specific RNA is hybridized to DNA, which is present in a sample. Upon hybridization, the reaction is treated with RNase H, and the products of the probe are identified as distinctive products, which are released after digestion. The original template is annealed to another cycling probe and the reaction is repeated. A number of other amplification processes are contemplated; however, the invention is not limited as to which method is used.

Following amplification of the Tey 1, it can be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al. (1989), supra.

Alternatively, chromatographic techniques can be employed to effect separation. There are many kinds of chromatography which can be used in the context of the present inventive methods e.g., adsorption, partition, ion-exchange and molecular sieve, and many specialized techniques for using them including column, paper, thin-layer and gas chromatography (Freifelder, Physical Biochemistry Applications to Biochemistry and Molecular Biology, 2nd Ed., Wm. Freeman and Co., New York, N.Y. (1982)).

Amplification products must be visualized in order to confirm amplification of the Tey 1 sequence. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified Tey 1 sequence. The probe preferably is conjugated to a chromophore but may be radiolabeled. In another embodiment, the probe is conjugated to a binding partner, such as an antibody or biotin, where the other member of the binding pair carries a detectable moiety (i.e., a label).

One example of the foregoing is described in U.S. Pat. No. 5,279,721, which discloses an apparatus and method for the automated electrophoresis and transfer of nucleic acids. The apparatus permits electrophoresis and blotting without external manipulation of the gel and is ideally suited to carrying out methods according to the present invention.

It will be understood that the probes described above are limited in as much as any nucleic-acid sequence can be used as long as the nucleic acid sequence is hybridizable to nucleic acids encoding Tey 1 or functional sequence analogs thereof. For example, a nucleic acid of partial sequence can be used to quantify the expression of a structurally related gene or the full-length genomic or cDNA clone from which it is derived.

Preferably, the hybridization is done under high stringency conditions. By “high stringency conditions” is meant that the probe specifically hybridizes to a target sequence in an amount that is detectably stronger than non-specific hybridization. High stringency conditions, then, would be conditions which would distinguish a polynucleotide with an exact complementary sequence, or one containing only a few scattered mismatches from a random sequence that happened to have a few small regions (e.g., 3-10 bases) that matched the probe. Such small regions of complementarity, are more easily melted than a full-length complement of 14-17 or more bases and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the probe and the template or target strand, and are particularly suitable for detecting expression of specific Tey 1. It is generally appreciated that conditions can be rendered more stringent by the addition of increasing amounts of formamide.

When a Tey 1 protein or polypeptide fragment thereof is assayed, various assays (i.e., immunobinding assays) are contemplated to detect or measure the quantity of Tey 1. In such embodiments, the Tey 1 can be employed to detect antibodies having reactivity therewith, or, alternatively, antibodies can be prepared and employed to detect the Tey 1. The steps of various useful immunodetection assays have been described in Nakamura et al., Handbook of Experimental Immunology (4th Ed.), Wol. 1, Chapter 27, Blackwell Scientific Publ., Oxford (1987); Nakamura et al., Enzyme Immunoassays: Heterogenous and Homogenous Systems, Chapter 27 (1987) and include Western hybridization (i.e., Western blots), immunoaffinity purification, immunoaffinity detection, enzyme-linked immunosorbent assay (e.g., an ELISA), and radioimmunoassay. A microarray also can be used to detect or measure the levels of Tey 1.

In general, the immunobinding assays involve obtaining a test sample suspected of containing a protein, peptide or antibody corresponding to a Tey 1, and contacting the sample with an antibody in accordance with the present invention, as the case may be, under conditions effective to allow the formation of immunocomplexes. Indeed, a mammal can be diagnosed with a cancer by either detecting Tey 1 or an antibody that recognizes Tey 1, or by quantifying the levels of Tey 1 or an antibody that recognizes Tey 1. Any suitable antibody can be used in conjunction with the present invention such that the antibody is specific for Tey 1.

The immunobinding assays for use in the present invention include methods for detecting or quantifying the amount of Tey 1 in a sample, which methods require the detection or quantitation of any immune complexes formed during the binding process. Here, a test sample suspected of containing a Tey 1 would be obtained from a mammal and subsequently contacted with an antibody (e.g., MHS-5). The detection or the quantification of the amount of immune complexes formed under the specific conditions is then performed.

Contacting the biological sample with an antibody that recognizes a Tey 1 under conditions effective and for a period of time sufficient to allow formation of immune complexes (primary immune complexes) is generally a matter of simply adding the antibody to the sample and incubating the mixture for a period of time long enough for the antibodies to form immune complexes with, i.e., to bind to, any antigens. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well-known in the art and can be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837, 3,850,752, 3,939,350, 3,996,345, 4,277,437, 4,275,149 and 4,366,241. Of course, additional advantages can be realized by using a secondary binding ligand, such as a second antibody or a biotin/avidin ligand binding arrangement, as is known in the art.

The antibody which is used in the context of the present invention can, itself, be linked to a detectable label, wherein one would then simply detect this label, thereby allowing the presence of or the amount of the primary immune complexes to be determined.

Alternatively, the first added component that becomes bound within the primary immune complexes can be detected by means of a second binding ligand that has binding affinity for the first antibody. In these cases, the second binding ligand is, itself, often an antibody, which can be termed a “secondary” antibody. The primary immune complexes are contacted with the labeled, secondary binding ligand, or antibody, under conditions effective and for a period of time sufficient to allow the formation of secondary immune complexes. The secondary immune complexes are then washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the first antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed.

It will be understood that other diagnostic tests can be used in conjunction with the diagnostic tests described herein to enhance further the accuracy of diagnosing a mammal with a cancer. For example, a monoclonal antibody, such as the ones described in U.S. Pat. No. 4,569,788, can be used effectively in diagnosing small-cell lung cancer over non small-cell lung cancer.

A method of prognosticating cancer in a mammal is also provided. The method comprises (a) obtaining a test sample from the mammal, and (b) assaying the test sample for the level of Tey 1. The level of Tey 1 in the test sample can be measured by comparing the level of Tey 1 in another test sample obtained from the mammal over time in accordance with the methods described above. An increase in the level of Tey 1 over time is indicative of a positive prognosis and a decrease in the level of Tey 1 over time is indicative of a negative prognosis. The method can be used to assess the efficacy of treatment of the cancer.

### EXAMPLE

The following example serves to illustrate the present invention and is not intended to limit its scope in any way.

The following references, to the extent that they provide exemplary procedural or other details supplementary to those set forth herein, are specifically incorporated herein by reference:
Birren et al., Genome Analysis: A Laboratory Manual Series, Volume 1, Analyzing DNA, Cold Spring Harbor Laboratory Press, Cold Spring Harbor, N.Y. (1997),
Birren et al., Genome Analysis: A Laboratory Manual Series, Volume 2, Detecting Genes, Cold Spring Harbor Laboratory Press, Cold Spring Harbor, N.Y. (1998),
Birren et al., Genome Analysis: A Laboratory Manual Series, Volume 3, Cloning Systems, Cold Spring Harbor Laboratory Press, Cold Spring Harbor, N.Y. (1999),
Birren et al., Genome Analysis: A Laboratory Manual Series, Volume 4, Mapping Genomes, Cold Spring Harbor Laboratory Press, Cold Spring Harbor, N.Y. (1999),
Harlow et al., Antibodies: A Laboratory Manual, Cold Spring Harbor Laboratory Press, Cold Spring Harbor, N.Y. (1988),
Harlow et al., Using Antibodies: A Laboratory Manual, Cold Spring Harbor Laboratory Press, Cold Spring Harbor, N.Y. (1999),
Hoffman, Cancer and the Search for Selective Biochemical Inhibitors, CRC Press (1999),
Pratt, The Anticancer Drugs, 2nd edition, Oxford University Press, NY (1994), and
Sambrook et al., Molecular Cloning. A Laboratory Manual, 2nd edition, Cold Spring Harbor Laboratory Press, Cold Spring Harbor, N.Y. (1989).

### Example 1

This example describes the cloning and sequencing of the Tey 1.

A 60 Kb bacterial artificial chromosome (BAC) clone on human chromosome 8, encoding a metastasis suppressor gene for highly metastatic rat prostate cancer cell line AT6.3, was identified (Nihei et al., Cancer Research (2001), accepted). Several techniques were used to identify genes in this region, including cDNA selection (Morgan et al., Nucleic Acids Research 20: 5173-5179 (1992)) and genomic sequencing of the BAC clone Platzer et al., Genome Research 7: 592-605 (1997)).

cDNA selection was performed as described (Morgan et al. (1992), supra). Poly (A)+ RNA was isolated from a microcell hybrid (AT6.3-8-22) which contained the metastasis suppressor gene (Sambrook et al. (1989), supra) using a poly(A) pure mRNA isolation kit (Ambion, Austin, Tex.). Normal human prostate poly (A)+ RNA (Clontech, Palo Alto, Calif.) was also used separately. Random-primed double stranded cDNA was synthesized from 5 μg of these RNAs using SuperScript Choice System for cDNA Synthesis (Life Technologies, Grand Island, N.Y.), digested with Sau 3A1, ligated to Mbo linker I, and amplified by PCR as described (Sambrook et al., (1989), supra). BAC DNA was isolated using NucleoBond BAC maxi kit (Clontech), digested with Sau 3A1, ligated to Mbo linker II, and amplified by PCR with 5′ biotinylated primer as described (Morgan et al. (1992), supra). After purification and size selection by PCR purification kit (Qiagen, Valencia, Calif.), repeat sequences in both the biotin-labeled selector DNA and the target cDNA were suppressed by annealing with CotI DNA (Life Technologies). the selector BAC DNA (100 ng) and the target cDNA (1 μg) were hybridized at 65° C. in a 100 μl solution of 6×SSC and 0.1% SDS for 16 hr. Selected cDNAs were captured with streptavidin-conjugated paramagnetic particles (Promega, Madison, Wis.), eluted in 50 μl of 50 mM NaOH, and amplified by PCR as described (Morgan et al. (1992), supra). These amplified eluted cDNAs were recycled through the above process for secondary selection. Second-round selected cDNAs were digested with Eco RI, and ligated into Eco RI-digested and phosphatased pUC18 plasmid vector. These cloned cDNA inserts were then transformed into XL-10 gold ultracompetent cells (Stratagene, La Jolla, Calif.). Colony PCR was performed on 400 white colonies with M13/pUC sequencing primers (Life Technologies), and the PCR products were blotted on Hybond N+ nylon membranes (Amersham Pharmacia, Piscataway, N.J.). These blots were hybridized with the 60 Kb BAC DNA and no insert BAC DNA by standard methods (Sambrook et al. (1989), supra). Approximately 100 clones were mapped back to the 60 Kb BAC clone and sequenced using dRhodamine cycle sequencing kit (Applied Biosystems, Foster City, Calif.) on an ABI 377 automated sequencer (Applied Biosystems). Sequences of transcripts obtained by cDNA selection were verified against the genomic sequence of the 60 Kb BAC clone.

Genomic sequencing of the 60 Kb BAC clone was performed by the shotgun method (Platzer et al., Genome Research 7: 592-605 (1997)) at the NIH Intramural Sequencing Center. The genomic sequence information was used to identify expressed sequence tags (ESTs) using the BLAST program (Altschul et al., J. Mol. Biol. 215: 403-410 (1990)), and to perform gene screening using GRAIL and GENSCAN gene prediction programs (Uberbacher et al., PNAS USA 88: 11261-11265 (1991)).

These analyses identified a single novel gene within this region. In order to isolate a full-length cDNA, three different cDNA library screening methods were used. Traditional plaque filter hybridization with an adult human prostate cDNA library (Clontech) and kidney cDNA library (Stratagene) was performed by standard methods (Altschul et al. (1990), supra). A cDNA library using poly(A)+ RNA isolated from the AT6.3 rat prostate cancer cells transfected with the 60 Kb BAC clone (SuperScript Choice System for cDNA Synthesis, Life Technologies) also was constructed and used for the third screening. The accuracy of the cDNA sequences was confirmed by RT-PCR and Northern blot analysis using gene-specific primer pairs and probes by standard methods (Altschul et al. (1990), supra).

Direct comparison of the genomic sequence of the 60 Kb BAC clone and the cDNA sequences allowed the exon-intron boundaries to be determined for all exons.

All of the references cited herein, including patents, patent applications, and publications, are hereby incorporated in their entireties by reference.

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

What is claimed is:

1. An isolated or purified nucleic acid molecule consisting essentially of a nucleotide sequence encoding the metastasis suppressor gene located at p21-p12 on chromosome 8 of a human (Tey 1) or a fragment thereof comprising at least 455 contiguous nucleotides.

2. The isolated or purified nucleic acid molecule of claim 1, which (i) encodes the amino acid sequence of SEQ ID NO: 3 or 5, (ii) consists essentially of the nucleotide sequence of SEQ ID NO: 1, 2 or 4 or a fragment thereof comprising at least 455 contiguous nucleotides, (iii) hybridizes under low stringency conditions to an isolated or purified nucleic acid molecule consisting essentially of the nucleotide sequence that is complementary to SEQ ID NO: 1, 2 or 4 or a fragment thereof comprising at least 455 contiguous nucleotides, or (iv) shares 50% or more identity with SEQ ID NO: 1, 2 or 4.

3. An isolated or purified nucleic acid molecule consisting essentially of a nucleotide sequence encoding a variant Tey 1, which comprises one or more insertions, deletions, substitutions, and/or inversions, wherein the variant Tey 1 encoded by the isolated or purified nucleic acid molecule does not differ functionally from the corresponding unmodified Tey 1, or a fragment thereof comprising at least 455 contiguous nucleotides.

4. The isolated or purified nucleic acid molecule of claim 3, wherein the variant Tey 1 is able to suppress metastasis of a highly metastatic prostatic tumor cell line in vivo at least about 90% as well as the unmodified Tey 1 comprising SEQ ID NO: 3 or 5.

5. An isolated or purified nucleic acid molecule consisting essentially of a nucleotide sequence that is complementary to a nucleotide sequence encoding Tey 1 or a fragment thereof comprising at least 455 contiguous nucleotides.

6. The isolated or purified nucleic acid molecule of claim 5, which (i) is complementary to a nucleotide sequence encoding the amino acid sequence of SEQ ID NO: 3 or 5, (ii) is complementary to the nucleotide sequence of SEQ ID NO: 1, 2 or 4 or a fragment thereof comprising at least 455 contiguous nucleotides, (iii) hybridizes under low stringency conditions to an isolated or purified nucleic acid molecule consisting essentially of SEQ ID NO: 1, 2 or 4 or a fragment thereof comprising at least 455 contiguous nucleotides, or (iv) shares 50% or more identity with the nucleotide sequence that is complementary to SEQ ID NO: 1, 2 or 4.

7. An isolated or purified nucleic acid molecule consisting essentially of a nucleotide sequence that is complementary to a nucleotide sequence encoding a variant Tey 1.

8. A vector comprising the isolated or purified nucleic acid molecule of claim 1, optionally as part of an encoded fusion protein.

9. A vector comprising the isolated or purified nucleic acid molecule of claim 2, optionally as part of an encoded fusion protein.

10. A vector comprising the isolated or purified nucleic acid molecule of claim 3, optionally as part of an encoded fusion protein.

11. A vector comprising the isolated or purified nucleic acid molecule of claim 4, optionally as part of an encoded fusion protein.

12. A vector comprising the isolated or purified nucleic acid molecule of claim 5.

13. A vector comprising the isolated or purified nucleic acid molecule of claim 6.

14. A vector comprising the isolated or purified nucleic acid molecule of claim 7.

15. A cell comprising and expressing the isolated or purified nucleic acid molecule of claim 1, optionally in the form of a vector.

16. A cell comprising and expressing the isolated or purified nucleic acid molecule of claim 2, optionally in the form of a vector.

17. A cell comprising and expressing the isolated or purified nucleic acid molecule of claim 3, optionally in the form of a vector.

18. A cell comprising and expressing the isolated or purified nucleic acid molecule of claim 4, optionally in the form of a vector.

19. A cell comprising and expressing the isolated or purified nucleic acid molecule of claim 5, optionally in the form of a vector.

20. A cell comprising and expressing the isolated or purified nucleic acid molecule of claim 6, optionally in the form of a vector.

21. A cell comprising and expressing the isolated or purified nucleic acid molecule of claim 7, optionally in the form of a vector.

22. An isolated or purified polypeptide molecule consisting essentially of an amino acid sequence encoding Tey 1 or at least 6 contiguous amino acids of Tey 1, which is optionally glycosylated, amidated, carboxylated, phosphorylated, esterified, N-acylated or converted into an acid addition salt.

23. A conjugate or fusion protein comprising the isolated or purified polypeptide molecule of claim 22 and a therapeutically or prophylactically active agent.

24. The conjugate of claim 23, wherein the therapeutically or prophylactically active agent is an anti-cancer agent.

25. A composition comprising the isolated or purified polypeptide molecule of claim 22, optionally in the form of a conjugate or a fusion protein comprising a therapeutically or prophylactically active agent, and an excipient or an adjuvant.

26. An isolated or purified polypeptide molecule consisting essentially of an amino acid sequence encoding a variant Tey 1 or at least 6 contiguous amino acids of a variant Tey 1, which is optionally glycosylated, amidated, carboxylated, phosphorylated, esterified, N-acylated or converted into an acid addition salt.

27. A conjugate or fusion protein comprising the isolated or purified polypeptide molecule of claim 26 and a therapeutically or prophylactically active agent.

28. The conjugate of claim 27, wherein the therapeutically or prophylactically active agent is an anti-cancer agent.

29. A composition comprising the isolated or purified polypeptide molecule of claim 26, optionally in the form of a conjugate or a fusion protein comprising a therapeutically or prophylactically active agent, and an excipient or an adjuvant.

30. A method of treating cancer prophylactically or therapeutically in a mammal, which method comprises administering to the mammal an effective amount of:
(a) an isolated or purified nucleic acid molecule encoding Tey 1, optionally in the form of a vector, or
(b) an isolated or purified Tey 1 polypeptide, optionally in the form of a conjugate or fusion protein,
whereupon the mammal is treated for the cancer prophylactically or therapeutically.

31. The method of claim 30, wherein the cancer is prostate cancer.

32. A method of diagnosing cancer in a mammal, which method comprises:
(a) obtaining a test sample from the mammal, and
(b) assaying the test sample for the level of Tey 1, wherein a decrease in the level of Tey 1 in the test sample as compared to the level of Tey 1 in a control sample is diagnostic for the cancer.

33. A method of prognosticating cancer in a mammal, which method comprises:
(a) obtaining a test sample from the mammal, and
(b) assaying the test sample for the level of Tey 1, wherein an increase in the level of Tey 1 over time is indicative of a positive prognosis and a decrease in the level of Tey 1 over time is indicative of a negative prognosis.

34. The method of claim 33, wherein the method is used to assess the efficacy of treatment of the cancer.
